(19) United States
(12) Patent Application Publication
(10) Pub. No.: US 2024/0366885 A1
(43) Pub. Date: Nov. 7, 2024

(54) MEDICATION DELIVERY DEVICES, SYSTEMS AND METHODS

(71) Applicant: Bigfoot Biomedical, Inc., Milpitas, CA (US)

(72) Inventors: Rachael Price, Milpitas, CA (US); Matthew Clemente, Milpitas, CA (US); Laurie Mendelson, Milpitas, CA (US); Apurva Jadhav, Milpitas, CA (US); Horst Pitchler, Milpitas, CA (US); Amy Connell, Milpitas, CA (US); Matthew Johnson, Milpitas, CA (US); Namisha Lnu, Milpitas, CA (US); Christina Bode, Milpitas, CA (US); Ian Hanson, Milpitas, CA (US); Aaron Morgan, Milpitas, CA (US); Jane Zhang, Milpitas, CA (US); Jenny Nguyen, Milpitas, CA (US); Pawel Drozdz, Milpitas, CA (US); Sarah Vaughan, Milpitas, CA (US); Scott Telstad, Milpitas, CA (US); Olga Smelyanets, Milpitas, CA (US); Jorge A. Balderrama Canales, Milpitas, CA (US); Janet Wu Chenie Chastain, Milpitas, CA (US); Thomas Dean Jordan, Milpitas, CA (US); Corydon A. Hinton, Milpitas, CA (US); John Fresquez, Milpitas, CA (US); David Lane, Milpitas, CA (US); Chris Puryear, Milpitas, CA (US); Peter Kimball, Milpitas, CA (US); Vidya Saptharishi, Milpitas, CA (US); Paul Pasika, Milpitas, CA (US); Ashwin Sriram, Milpitas, CA (US)

(21) Appl. No.: 18/654,826

(22) Filed: May 3, 2024

Related U.S. Application Data
(60) Provisional application No. 63/500,362, filed on May 5, 2023.

(51) Int. Cl. A61M 5/32

(52) U.S. Cl. CPC A61M 5/326; A61M 5/3204; A61M 2005/3247

(57) ABSTRACT

Medication delivery methods, devices and systems are provided. The medication delivery device includes a retractable sheath mechanism connected to a distal end of the device. The retractable sheath mechanism is axially movable between a first position to cover a needle attached to the distal end and a second position to expose the needle with an adjustable protrusion length. The retractable sheath mechanism includes one or more skin sensors disposed on an end surface thereof to detect skin before and during injection. In some cases, a needle carousel is provided to connect to the distal end of the device. The needle carousel supports multiple needle capsules each including a needle.

### TECHNICAL FIELD

This disclosure relates generally to medication delivery devices and methods. More specifically, the disclosure relates to injection devices and methods for delivering medication via needles.

### BACKGROUND

Diabetes treatment devices (e.g., glucose meters, insulin pumps, insulin injection devices, etc.) are widely used by a person with diabetes (PWD) to self-administer external biologically effective drugs (e.g., insulin or its analog). In general, needles are manually attached to injection devices and there is a need for sharps disposal of spent needles.

### SUMMARY

This disclosure relates generally to medication delivery devices and methods. More specifically, the disclosure relates to injection devices and methods for delivering medication via needles.

Briefly, in one embodiment, the present disclosure describes a medication delivery device including a housing extending along a housing axis between a distal end and a proximal end thereof, and a needle cover connected to the distal end of the housing. The needle cover includes a retractable sheath mechanism axially movable between a first position and a second position, and one or more skin sensors disposed on an end surface thereof to detect skin.

In another embodiment, the present disclosure describes a method of using a medication delivery device. The method includes receiving, via a user interface of the medication delivery device, an instruction to deliver medication into a user's skin, and upon receiving the instruction, measuring, via one or more skin sensors, a contact between a retractable sheath mechanism of the medication delivery device and the user's skin. The retractable sheath mechanism retracts from a first position to a second position to allow a needle of the medication delivery device to insert into the user's skin. The method further includes determining, via a controller of the medication delivery device, whether the needle is inserted into the user's skin with a predetermined injection depth based on the measured contact. The method further includes determining, via a controller of the medication delivery device, that the needle is inserted into the user's skin with a predetermined injection depth based on the measured contact. Upon determining that the needle of the medication delivery device is inserted into the user's skin with the predetermined injection depth, delivering medication into the user's skin.

In another embodiment, the present disclosure describes a method of using a medication delivery device. The method includes providing a needle carousel to support a plurality of needle capsules disposed around a carousel axis thereof, each needle capsule including a needle and a needle spring to suspend the needle between opposite ends of the respective needle capsules. The method further includes rotating the needle carousel about the carousel axis to align the needle of the respective needle capsules to an injection end of the medication delivery device, engaging the needle with the injection end of the medication delivery device, extending the needle to project from a distal end of the respective needle capsules, and retracting the needle into the respective needle capsules. In some cases, the device is automatically primed before extending the needle.

Various aspects and advantages of exemplary embodiments of the disclosure have been summarized. The above Summary is not intended to describe each illustrated embodiment. Other features and aspects will become apparent by consideration of the following detailed description and accompanying drawings.

### BRIEF DESCRIPTION OF THE DRAWINGS

References are made to the accompanying drawings that form a part of this disclosure and which illustrate the embodiments in which systems and methods described in this specification can be practiced.

FIG. 1A is a side perspective view of a medication delivery device, according to an embodiment.

FIG. 1B is a cross-sectional view of the medication delivery device of FIG. 1A.

FIG. 2A is a cross-sectional view of a medication delivery device with a needle cover at a first position, according to an embodiment.

FIG. 2B is a cross-sectional view of the medication delivery device of FIG. 2A with the needle cover at a second position.

FIG. 3 is a flow diagram of a method of using a medication delivery device, according to an embodiment.

FIG. 4A is a schematic diagram of a portion of a medication delivery device including a needle cover, according to an embodiment.

FIG. 4B is a cross-sectional view of the needle cover of FIG. 4A.

FIG. 4C is a side perspective view of an inside of an outer collar of the needle cover of FIG. 4A.

FIG. 4D is a side perspective view of an outside of the outer collar of the needle cover of FIG. 4A

FIG. 4E is a side perspective view of an inner collar of the needle cover of FIG. 4A.

FIG. 5A is an exploded view of a medication delivery device, according to an embodiment.

FIG. 5B-5F illustrate side perspective views of a clicker mechanism, according to an embodiment.

FIG. 5G is an end perspective view of the medication delivery device of FIG. 5A without a carousel cover in place.

FIG. 5H is an end perspective view of the medication delivery device of FIG. 5A with a carousel cover in place.

FIG. 6 is a cross-sectional view of a needle capsule, according to an embodiment.

FIG. 7 is a block diagram illustrating a medication delivery device under different stages of operation, according to an embodiment.

Particular embodiments of the present disclosure are described herein with reference to the accompanying drawings; however, it is to be understood that the disclosed embodiments are merely examples of the disclosure, which may be embodied in various forms. Well-known functions or constructions are not described in detail to avoid obscuring the present disclosure in unnecessary detail. Therefore, specific structural and functional details disclosed herein are not to be interpreted as limiting, but merely as a basis for the claims and as a representative basis for teaching one skilled in the art to variously employ the present disclosure in virtually any appropriately detailed structure. In this description, as well as in the drawings, like-referenced numbers represent like elements that may perform the same, similar, or equivalent functions.

### DETAILED DESCRIPTION

This disclosure relates generally to medication delivery devices and methods. More specifically, the disclosure relates to devices and methods for delivering medication to a user, in particular, to injection devices, methods for using injection devices, and methods for delivering medication via needles.

Systems, devices and methods described herein can be applied to deliver various medications and manage various diseases including, for example, diabetes, blood thinners such as Heparin, palliative care pain medications, growth hormone deficiency treatment such as somatotropin, fertility treatment such as human chorionic gonadotropin or follicle stimulating hormone, etc. In some embodiments, systems, devices and methods described herein can provide multiple, daily delivery of medication, for example, a mixture of rapid- and long-acting insulin via a medication delivery device to a user. The systems, devices and methods allow a user (e.g., a person with diabetes or PWD) to safely inject medication into tissues without the concern of frequently changing needles and sharps disposal of spent needles.

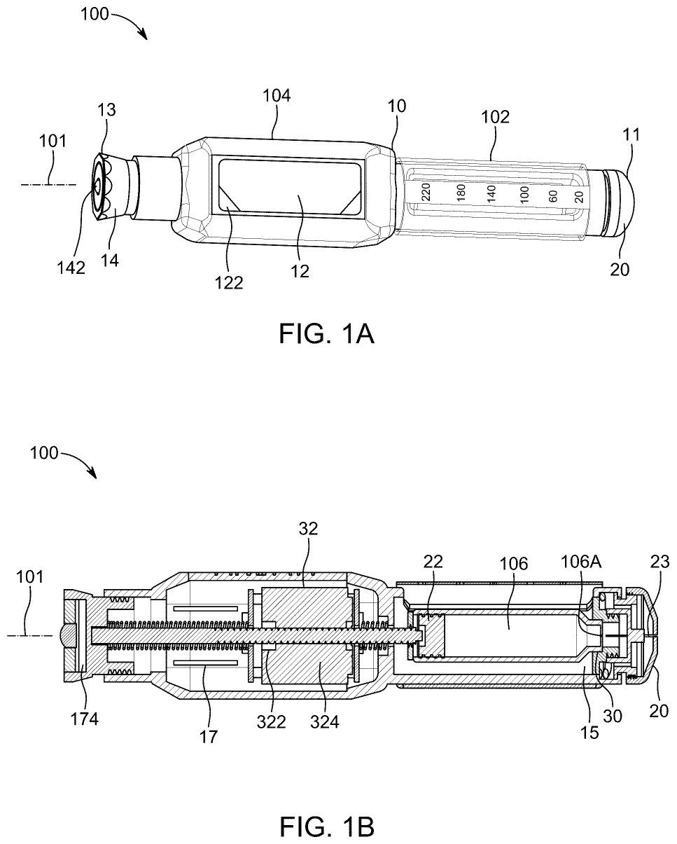

FIG. 1A is a side perspective view of a medication delivery device 100, according to an embodiment. FIG. 1B is a cross-sectional view of the medication delivery device 100 of FIG. 1A. The medication delivery device 100 includes a housing 10 extending along a housing axis 101 between a first end 11 (e.g., a distal end) and a second end 13 (e.g., a proximal end) thereof in a longitudinal direction. The term “distal” may refer to being situated away from a user (e.g., the user's hand handling the device during the operation). A distal end of a device may refer to an end of the device that is away from the user's hand during the operation. Accordingly, a proximal end of a device may refer to an end of the device that is close to the user's hand during the operation.

The housing 10 includes a first part 102 at the first end 11 and a second part 104 at the second end 13. The first part 102 defines an internal space to receive a medication cartridge 106, to which a needle 23 is attached at the first end 11 of the housing 10. The medication cartridge 106 can be inserted into the first part 102 of the housing 10 via an opening 15 at the first end 11 of the housing 10 along the longitudinal direction as indicated by the housing axis 101. The medication cartridge 106 can be positioned inside the housing 10, with a distal end 106a being accessible from the first end 11 of the housing 10 to mount the needle 23 thereon via a needle adapter 30. The distal end 106a of the medication cartridge 106 may be hermetically sealed by, e.g., a rubber stopper. The needle adapter 30 can be mounted at the first end 11 of the housing 10 to receive the distal end 106a of the medication cartridge 106 at one side and allow the needle 23 to extend from the opposite side to penetrate into the medication cartridge 106.

The first part 102 of the housing 10 may have a transparent or semi-transparent wall to allow a user to observe the medication cartridge 106 inside the housing 10. The medication cartridge 106 may contain multiple doses of medication and include a displacement member 22 disposed at least partially inside the medication cartridge 106. The displacement member 22 is movable along the longitudinal direction of the medication cartridge 106 toward the first end 11 of the housing 10 to deliver medication therefrom. The displacement member 22 can be, for example, a plunger. Exemplary insulin cartridges are commercially available from Novo Nordisk A/S (Bagsvaerd, Denmark) under the trade designation of NovoLog, and from Eli Lilly and Company (Indianapolis, Indiana) under the trade designation of HUMALOG.

In some cases, the housing 10 can have a one-piece construction, of which the first part 102 and the second part 104 can connect to each other by an engagement mechanism (e.g., a screwing mechanism). The medication cartridge 106 can be loaded into the first part 102 and ejected out via an ejection mechanism such as, for example, a spring loaded bayonet mechanism. The medication delivery device 100 further includes a display panel 12 disposed on an outer surface of the housing 10. The display panel 12 may include a display to present various information to a user, including, for example, analyte measurement data (e.g., continuous glucose monitor (CGM) data, flash glucose monitor data, glucose meter data, etc.), a query to a user to solicit the user to input parameters to run a drive mechanism to customize medication delivery, an instruction to the user to prime the medication delivery device before automatically delivering the intended dose, a feedback to the user to place the device on skin, a notification to the user to insert the needle, a feedback to the user to adjust a needle position, a notification to the user that the medication delivery is completed, a notification to the user that the medication delivery is not completed, etc.

In some cases, the display panel 12 can be a part of user interface (UI) of the medication delivery device 100 for the user to interact and communicate with the medication delivery device 100. The display panel 12 may further include a touch screen, one or more touch buttons 122, one or more smart sensors to detect a user's action (e.g., a finger touch), or other input mechanisms to receive the user's input. The one or more touch buttons on a touch screen may include, for example, an injection button, an ejection button, etc.

The medication delivery device 100 further includes one or more tactile buttons disposed on an outer surface of the second part 104 of the housing 10. For example, the medication delivery device 100 includes an injection button 14 which is in the form of a knob. The injection button 14 has a low power indicator 142 disposed on an end surface thereof. The injection button 14 may be used by the user to indicate that the user is ready for injection.

The medication delivery device 100 further includes a drive mechanism 32 disposed inside the second part 104 of the housing 10. In the depicted embodiment, the drive mechanism 32 includes an electrical stepper motor 322 and a lead screw 324 driven by the stepper motor 322. The lead screw 324 has one end engaged with the displacement member 22 (e.g., a plunger) of the medication cartridge 106 to advance the displacement member 22 forward inside the medication cartridge 106 when the medication cartridge 106 is received in the housing 10 of the medication delivery device 100.

In some cases, the lead screw 324 can be actuated by the electrical stepper motor 322 with a linear actuation of the lead screw 324. Each turn of the lead screw 324 can be synchronized with one or more micro-steps of the electrical stepper motor 322 corresponding to a certain amount of medication dispensing at a certain amount of time (e.g., units of insulin per second). In some cases, a moving distance of the lead screw 324, and a position of the displacement member 22 (e.g., a plunger) and a speed and direction of the movement, can be controlled by an encoder (not shown) integrated into the electrical stepper motor 322. The encoder can provide digital signals for monitoring and controlling the parameters of the drive mechanism 32 to a controller 17, which will be described below. It is to be understood that the drive mechanism may include any suitable mechanical or electromechanical components to advance the displacement member 22 forward inside the medication cartridge 106.

The controller 17 includes a printed circuit board assembly (PCBA) that may include various control circuitry or electronic components such as, for example, logic circuitry, drive controller circuitry, wireless communication circuitry, memory, etc. The printed circuit board assembly (PCBA) can be supported by the housing 10, powered by the battery 174, and functionally connected to other components of the medication delivery device 100 such as, for example, the drive mechanism 32, the display 12, the injection button 14, etc. The controller 17 can execute one or more algorithms stored in the memory to control the delivery of medication into a user's skin.

The controller 17 can receive real-time digital signals for monitoring and controlling the parameters from the drive mechanism 32 and implement pre-determined algorithms to administer medication delivery, including, for example, determining whether the device is properly primed, determining the delivered dosage, determining the dosage to be delivered, etc. The controller 17 may present real time dosage information on the display 12. In some cases, the controller 17 may allow a user to provide input, via the user interface, to adjust parameters to run the drive mechanism 32, and to program and personalize medication delivery. In some cases, the controller 17 may receive a user's input, via the injection button 14, that the user is ready for injection. The controller 17, based on the user's input, may control the drive mechanism 32 to advance the displacement member 22 within the medication cartridge 106 to inject medicine into the user's skin, as discussed herein.

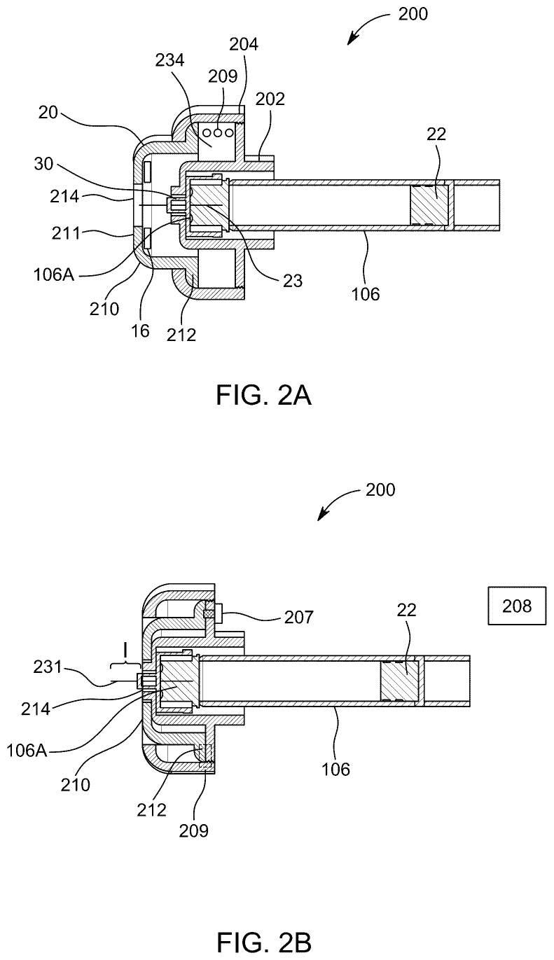

FIG. 2A is a cross-sectional view of a portion of a medication delivery device 200 with a needle cover 20 at a first position, according to an embodiment. FIG. 2B is a cross-sectional view of the medication delivery device 200 of FIG. 2A with the needle cover 20 at a second position. The medication delivery device 200 receives a medication cartridge 106 via a housing (e.g., the housing 10 in FIG. 1A). The needle 23 is attached to a distal end 106a of the medication cartridge 106 via a needle adaptor 30, and is at least partially covered by the needle cover 20. The medication cartridge 106 may be hermetically sealed, e.g., by a rubber stopper, at the end 106a, through which the needle 23 may penetrate into the medication cartridge 106.

The needle cover 20 includes an inner collar 202 to receive the distal end 106a of the cartridge 106, an outer collar 204 connected to the inner collar 202, and a sheath mechanism 210 movably connected to the outer collar 204. The outer collar 204 may be mounted at a distal end of the device housing (e.g., the first end 11 of the device 100 in FIGS. 1A-B). The sheath mechanism 210 has a base portion 212 received in a space 234 formed between the inner collar 202 and the outer collar 204. The sheath mechanism 210 at least partially covers the needle 23 mounted on the end 106a of the cartridge 106, and is movable between a first position (FIG. 2A) and a second position (FIG. 2B). When the sheath mechanism 210 is at the first position as shown in FIG. 2A, a distal tip 231 of the needle 23 is indented from an opening 214 of the sheath mechanism 210 such that the sheath mechanism 210 completely covers the needle 23 to limit the access to the needle 23 from outside. When the sheath mechanism 210 retracts from the first position to the second position as shown in FIG. 2B, the distal tip 231 of the needle 23 can project from the opening 214 of the sheath mechanism 210 with a protrusion length/suitable for inserting into a user's skin with a certain depth. The protrusion length/is measured from the distal tip 231 of the needle 23 to the opening 214 of the sheath mechanism 210. The protrusion length/may be in a range, for example, from about 1 mm to about 10 mm. In some cases, the protrusion length/may be adjustable for different injection depth as desired, which will be discussed further below.

When the needle 23 inserts into a user's skin, the sheath mechanism 210 has its edge surface 211 being pressed by the user's skin and retracts from the first position (FIG. 2A) to the second position (FIG. 2B). A locking mechanism 207 is provided to temporarily lock the sheath mechanism 210 at the second position as shown in FIG. 2B. The locking mechanism 207 may have a spring-loaded bayonet structure including a spring element 209. During the insertion of the needle 23, the spring element 209 changes from a neutral state to a compressed state. When the sheath mechanism 210 is at the second position, the locking mechanism 207 (e.g., the bayonet mechanism) can temporarily lock the sheath mechanism 210 at the second position. The term “temporarily” refers to the situation that the sheath mechanism 210 can be released from locking at the second position back to the first position when the needle insertion contact switch 208 is turned off. The locking mechanism 207 is configured to lock the base portion 212 of the sheath mechanism 210 at the second position when the spring element 209 is compressed by the base portion 212. At the same time, a needle insertion contact switch 208 can be automatically turned on upon the locking. The needle insertion contact switch 208 can have a combined configuration with the sheath mechanism 210 and a housing of the device 200. For example, in an embodiment, the sheath mechanism 210 may have a sensor slot (not shown) that connects into the housing of the medication delivery device 200. The needle insertion contact switch 208 may be activated through the slot in the sheath mechanism 210 which may connect into the medication delivery device. In another embodiment, the sheath mechanism 210 may utilize near-field communication (NFC) or other communication protocols to connect to the medication delivery device 200 to turn on/off the needle insertion contact switch 208.

The sheath mechanism 210 further includes one or more skin sensors 16 disposed on the edge surface 211 of the sheath mechanism 210. The skin sensors 16 are configured to detect a user's skin when the edge surface 211 of the sheath mechanism 210 is in close contact with the user's skin. In some cases, the skin sensors 16 may include one or more capacitive sensors configured to measure signal(s) and obtain information arising from a proximity interaction between the edge surface 211 and the user's skin. While a pair of skin sensors 16 are illustrated in FIG. 2A, it is to be understood that one or more skin sensors 16 can be used to detect the proximity of user's skin. In some cases, sensing data from multiple skin sensors 16 may be used to detect the orientation of the needle 23 with respect to the user's skin. For example, when the housing 10 is tilted with an angle such that the needle 23 is not straight into the skin, the skin sensors 16 may sense the angle and send out a signal indicating that the orientation needs to be adjusted. When the edge surface 211 and the user's skin are in an intimate contact with each other and the orientation is acceptable, the skin sensors 16 may send a signal to a controller (e.g., the controller 17 in FIG. 1B) to indicate that the medication delivery device 200 is at an acceptable position for medication delivery.

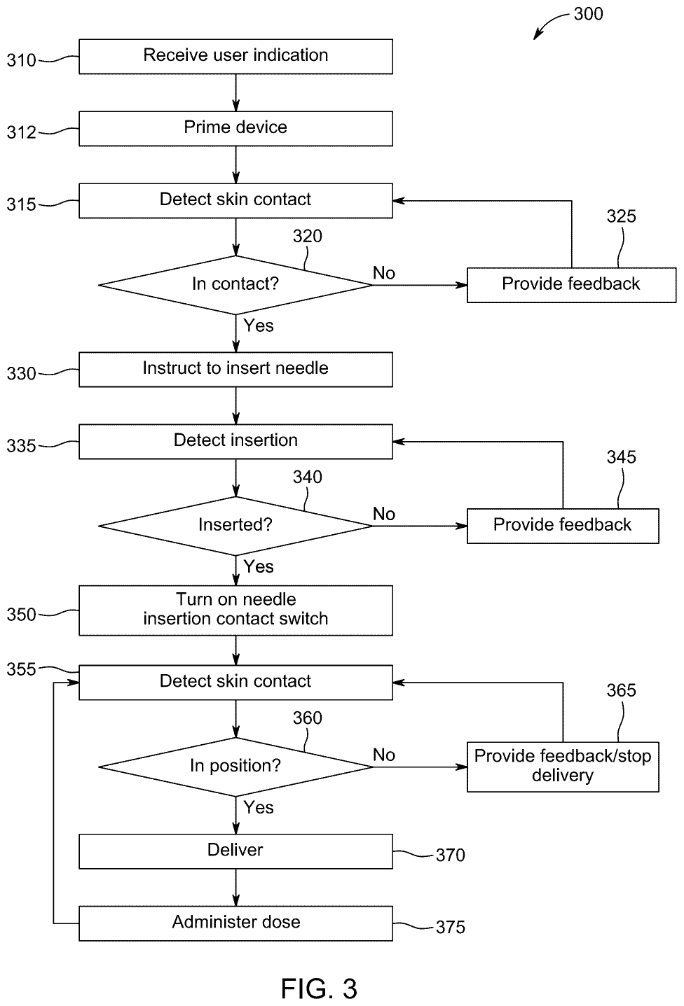

FIG. 3 is a flow diagram of a method 300 of using a medication delivery device, according to an embodiment. The method 300 may be employed to deliver medication using the medication delivery device. The medication delivery device can be any medication delivery device described herein such as, for example, the medication delivery device 100 in FIGS. 1A-B, or the medication delivery device 200 in FIGS. 2A-B. At block 310, the medication delivery device receives a user's indication that the user is ready for injection. The user's indication may be received, for example, via a user interface, injection button, etc. of the medication delivery device. The method 300 then proceeds to block 312.

At block 312, the medication delivery device automatically primes the device. Priming the medication delivery device prior to injection can remove air from the medication cartridge and the needle and prevent medication crystallization. The controller of the medication delivery device can instruct the drive mechanism to advance the displacement member within the medication cartridge with a pre-determined distance or units of dose. The distance or units of dose can be predetermined based on parameters such as, for example, a volume or size of the needle to be used. After the priming is completed, the medication delivery device may present a notification to the user and instruct the user to place the device on skin. The method 300 then proceeds to block 315.

At block 315, the skin sensor(s) 16 detect the user's skin when the edge surface 211 of the sheath mechanism 210 is in close contact with the user's skin. The skin sensor(s) 16 send sensing signals to the controller of the medication delivery device. The method 300 then proceeds to block 320.

At block 320, the controller of the medication delivery device processes the sensing signals from the skin sensor(s) 16 to determine whether the edge surface 211 of the sheath mechanism 210 is in close contact with the user's skin. When the controller of the medication delivery device determines that the edge surface 211 of the sheath mechanism 210 is not in close contact with the user's skin, the method 300 then proceeds to block 325. When the controller of the medication delivery device determines that the edge surface 211 of the sheath mechanism 210 is in close contact with the user's skin, the method 300 then proceeds to block 330.

At block 325, the medication delivery device presents, via the user interface, a feedback to the user to place the device on the skin or to adjust the position of the device on the skin. The medication delivery device may present the feedback on the display 12. The method 300 then proceeds back to block 315.

At block 330, the medication delivery device presents, via the user interface, an instruction to the user to insert the needle. For example, the medication delivery device may present the instruction on the display 12. It is to be understood that the user interface may deliver the instruction or notification to the user to insert the needle by any suitable means such as, a visual display, an audio notification, a tactile notification, etc. The method 300 then proceeds to block 335.

At block 335, the skin sensor(s) 16 detect the insertion of the needle into the skin. During the insertion of the needle into the skin, the spring element 209 changes from a neutral state to a compressed state, and the sheath mechanism 210 has its edge surface 211 being pressed by the user's skin and retracts from the first position (FIG. 2A) to the second position (FIG. 2B). The skin sensor(s) 16 may measure signal(s) and obtain information arising from the direct contact between the edge surface 211 and the user's skin. The skin sensor(s) 16 sense the direct contact with the user's skin during the process of insertion and send sensing signals to a controller of the medication delivery device. The method 300 then proceeds to block 340.

At block 340, the controller of the medication delivery device receives and processes the sensing signals from the skin sensor(s) 16 to determine whether the needle is inserted into the user's skin. When the controller of the medication delivery device determines that the needle is not inserted into the user's skin, the method 300 then proceeds to block 345. When the controller of the medication delivery device determines that the needle is inserted into the user's skin, the method 300 then proceeds to block 350.

At block 345, when the controller of the medication delivery device determines that the needle is not inserted into the user's skin, the medication delivery device presents, via the user interface, a feedback to the user to insert the needle into the skin. The method 300 then proceeds back to block 335.

At block 350, when the controller of the medication delivery device determines that the needle is inserted into the user's skin, the locking mechanism of the medication delivery device temporarily locks the sheath mechanism 210 at the second position as shown in FIG. 2B. At the same time, the medication delivery device can automatically turn on the needle insertion contact switch upon the locking. The method 300 then proceeds to block 355.

At block 355, the skin sensor(s) 16 keeps detecting the contact between the edge surface 211 of the sheath mechanism 210 and the user's skin the skin. When the needle is inserted into the user's skin and the sheath mechanism 210 is locked at the second position, the skin sensor(s) 16 keep sensing the close contact with the user's skin and sending sensing signals to the controller of the medication delivery device. The method 300 then proceeds to block 360.

At block 360, the controller of the medication delivery device receives and processes the sensing signals from the skin sensor(s) 16 to determine whether the needle is in position when the needle is inserted into the user's skin. For example, the controller can determine whether the needle is removed from the user's skin after the insertion. The controller can further determine whether the skin contact is maintained during the injection process. When the controller of the medication delivery device determines that needle is not in position with respect to the user's skin, the method 300 then proceeds to block 365. When the controller of the medication delivery device determines that the needle is in position with respect to the user's skin, the method 300 then proceeds to block 370.

At block 365, when the controller of the medication delivery device determines that needle is not inserted in position with respect to the user's skin, the medication delivery device presents, via the user interface, a feedback to the user to adjust the position of the needle with respect to the user's skin. The method 300 then proceeds back to block 355. When the medication delivery device is delivering medication into the user's skin and the controller determines that needle is not inserted in position with respect to the user's skin, the medication delivery device may stop the delivery and send a feedback to the user. For example, the controller of the medication delivery device may instruct the drive mechanism to stop running, and the medication delivery device may present, via the user interface, a notification to the user that the delivery is not completed.

At block 370, when the controller of the medication delivery device determines that the needle is inserted in position with respect to the user's skin, the medication delivery device delivers medication into the user's skin. For example, the controller of the medication delivery device may instruct the drive mechanism to advance the displacement member within the medication cartridge with a pre-determined distance or units of dose. The method 300 then proceeds to block 375.

At block 375, the medication delivery device administers, in real time, the delivery of medication. When the delivery is completed, the controller of the medication delivery device may instruct to turn off the needle insertion contact switch 208 to release the temporarily locked sheath mechanism 210 from the second position (see, e.g., FIG. 2B) back to the first position (see, e.g., FIG. 2A). The medication delivery device may further present, via the user interface a corresponding notification to the user. When the delivery is not completed, the method 300 then proceeds back to block 355. In this manner, during the process of injection, the skin sensor(s) can monitor the position of the needle inside the user's skin, and the medication delivery device can stop the injection when the controller determines that needle is not inserted in position.

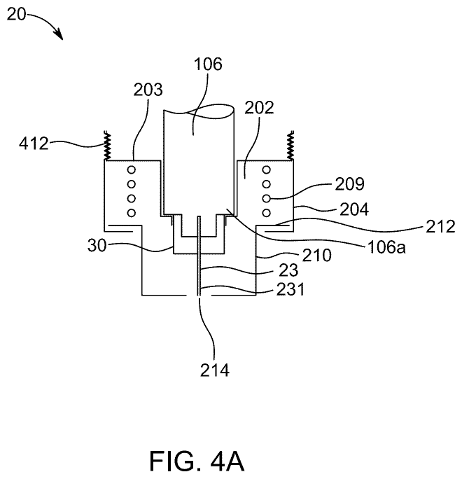

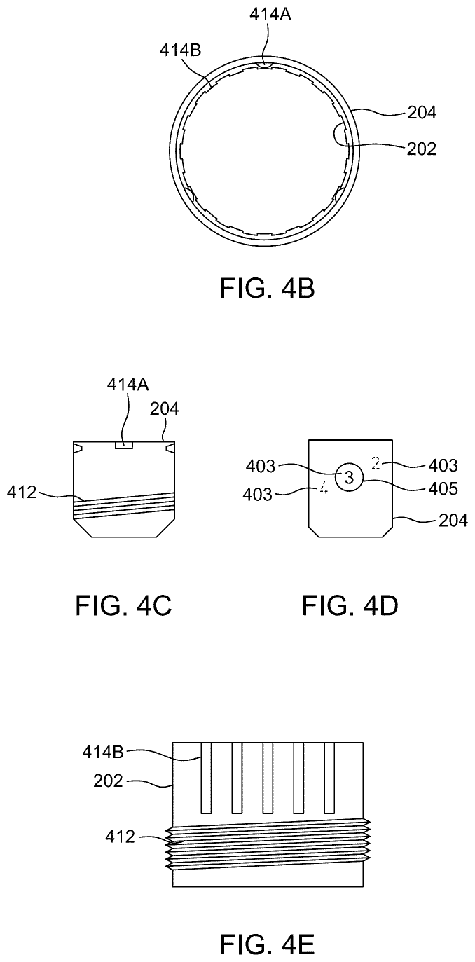

FIG. 4A is a schematic diagram of a needle cover 20 of a medication delivery device, according to an embodiment. FIG. 4B is a cross-sectional view of the needle cover 20 of FIG. 4A. The needle cover 20 includes an inner collar 202 to receive the distal end 106a of the medication cartridge 106, an outer collar 204 rotatably connected to the inner collar 202, and a sheath mechanism 210 movably connected to the outer collar 204. FIG. 4C is a side perspective view of the inside of the outer collar 204 of the needle cover 20. FIG. 4D is a side perspective view of the outside of the outer collar 204 of the needle cover 20. FIG. 4E is a side perspective view of the outside of the inner collar 202 of the needle cover 20.

At least one of the inner collar 202 and the outer collar 204 may be mounted at a distal end of the medication delivery device (e.g., the first end 11 of the medication delivery device 100 in FIGS. 1A-B). The spring element 209 is received in the space 234 formed between the inner collar 202 and the outer collar 204. The spring element 209 may have opposite ends disposed on a base portion 203 of the inner collar 203 and the base portion 212 of the sheath mechanism 210, respectively. The spring element 209 can be axially compressed to apply an outward bias against the sheath mechanism 210 to allow the sheath mechanism 210 to move between a first position (e.g., see FIG. 2A) and a second position (e.g., see FIG. 2B).

The outer collar 204 and the inner collar 202 are axially movable with respect to each other such that the relative axial positions of the needle 23 and the sheath mechanism 210 can be adjusted. The needle 23 is mounted on the needle adaptor 30, which may be attached to the inner collar 202. It is to be understood that the needle adaptor 30 may be a separate component that locks onto the cartridge 106. In the embodiment depicted in FIGS. 4A-E, threads 412 are provided to rotatably connect the outer collar 204 and the inner collar 202. When the outer collar 204 is rotated, via the threads 412, with respect to the inner collar 202, the sheath mechanism 210, along with the outer collar 204, can move axially with respect to the inner collar 202 and the needle 23. In this manner, the protrusion length (see, e.g., the protrusion length/as shown in FIG. 2B) of the needle 23 projecting out of the opening 214 of the sheath mechanism 210 can be adjusted. It is to be understood that the outer collar 204 and the inner collar 202 may have any suitable configurations to allow the relative axial movement with respect to each other to adjust the protrusion length of the needle 23.

As shown in FIGS. 4C-E, detent features 414a and 414b are provided on the inner surface of the outer collar 204 and the outer surface of the inner collar 202, respectively, to engage with each other when the outer collar 204 is rotated with respect to the inner collar 202. The detent features 414a-b are located to allow the sheath mechanism 210 to stop at discrete axial positions with respect to the needle 23 during the relative rotation of the outer collar 204 and the inner collar 202. The detent features 414a-b can have any suitable configurations to engage with each other to temporally lock the sheath mechanism 210 at the discrete axial positions, which may not be easily changed without a user's rotation of the outer collar 204 to overcome the engagement between the detent features 414.

The discrete axial positions of the sheath mechanism 210 can be labeled to create a reference for a user to set the desired insertion or injection depths of the needle 23. For example, labels 403 such as numerical numbers 2, 3, 4, etc., can be provided on the outer surface of the inner collar 202. The corresponding labels 403 can be visible, via a window 405 on the outer collar 204, to a user when the outer collar 204 and the inner collar 202 are rotated with respect to each other to allow the sheath mechanism 210 stop at the discrete axial positions.

In some cases, the needle cover 20 provides a dial for a user to turn one of the inner and outer collars to set the desired depths of needle insertion. The dial can have indications of pre-set depths (or protrusion lengths) of the needle 23 for insertion. The sheath mechanism 210 covers the needle 23 before insertion. When the sheath mechanism 210 is pressed against the user's skin, the needle 23 can travel the distance controlled by the sheath mechanism 210. The travel of the needle 23 relative to the sheath mechanism 210 can be created by pressing the sheath mechanism 210 against the user's skin, causing the sheath mechanism 210 to travel the pre-set distance, allowing the needle 23 to insert into the user's skin at customized depths. In some cases, the sheath mechanism 210 can be pressed against the user's skin and the medication cartridge 106 with the attached needle 23 can travel relative to the sheath mechanism 210 to a pre-set distance, allowing the needle 23 to insert into the user's skin with the customized depth.

Some embodiments described herein provide the customization of needle depth for injection of liquid through injection needle(s) of a medication delivery device. Needles for certain medication delivery devices often are available in different gauges and lengths, and a “box” of needles is typically packaged to have the same size (e.g., a needle length). There is a desire for a user to set a customized depth of needle injection for the needles having the same size. The depth of needle injection may be customized from injection to injection. In some embodiments described herein, the customization of needle depth provides the ability to adjust injection depth into tissue, without changing the size of needles. For example, the sheath mechanism 210 of FIG. 4A can set the needle depth in a range, for example, from at or about 1 mm to at or about 4 mm.

Adjustable depth of injection for one needle size can ensure a consistent amount of liquid for priming the needle. Priming a medication delivery device prior to injection can remove air from the medication cartridge and the needle and prevent medication crystallization. Needles having different sizes may need priming with different parameters. There is a desire to automatic priming for different injection depths without manually changing priming parameters. Some embodiments described herein provide adjustable depth of injection for one needle size, which can provide automatic priming of the needles by pre-setting the same parameters for priming the device using the same needle size. Eliminating needle size variability for priming can provide a clear expectation of product performance across user groups.

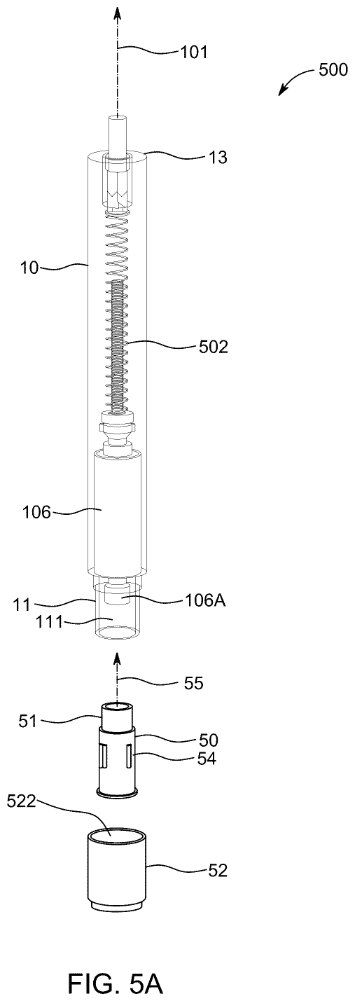

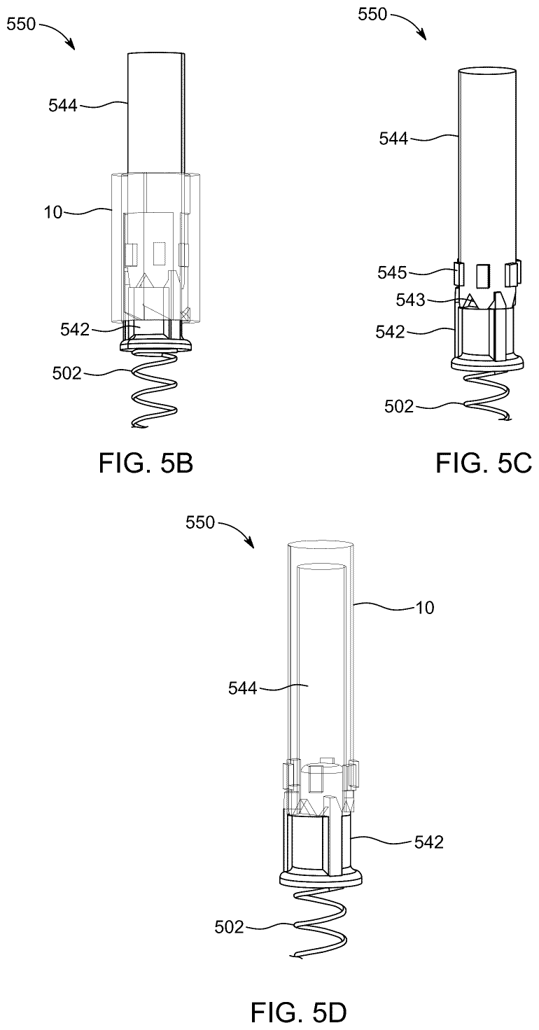

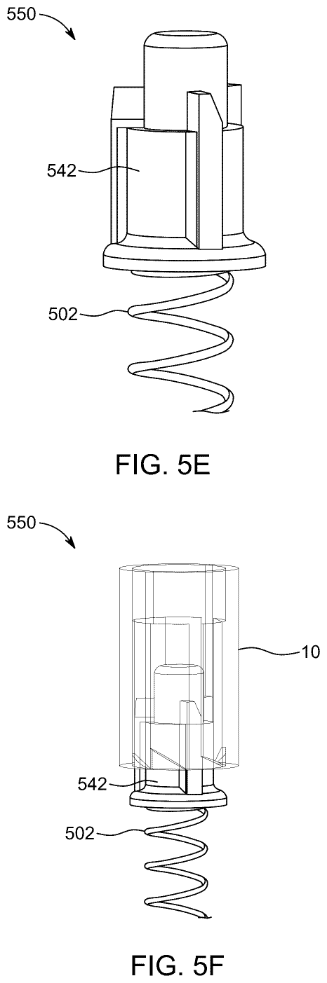

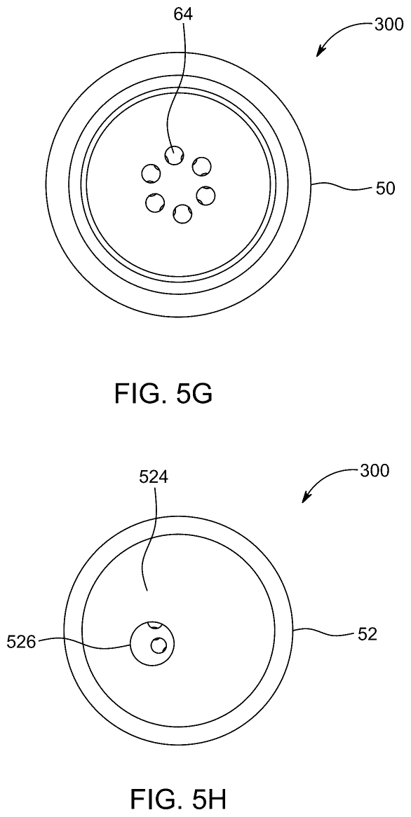

FIG. 5A is an exploded view of a medication delivery device 500, according to an embodiment. The medication delivery device 500 includes a housing 10 to receive the medication cartridge 106. A cartridge spring 502 is provided to compress against the medication cartridge 106 toward the first end 11 of the housing 10. A clicker mechanism 550 is provided to control the state of the cartridge spring 502. An embodiment of the clicker mechanism 550 is illustrated in FIGS. 5B-F. A needle carousel or drum 50 is attached to the first end 11 of the housing 10. A carousel/drum cover 52 is provided to cover the needle carousel or drum 50 when attached to the housing 10. FIGS. 5G-H are an end perspective view (from the side of carousel/drum cover 52) of the medication delivery device 500 of FIG. 5A without or with the carousel/drum cover 52 in place.

The clicker mechanism 550 includes a cam body 542 and a plunger 544 to engage with the cam body 542 via teeth or feet features 543. Engaging features 545 are also provided to engage with an inner wall of the housing 10. When the plunger 544 is pressed once, the spring 502 retracts at a retracted position as shown in FIG. 5B. When the clicker mechanism is pressed again, the spring 502 is extended at an extended position as shown in FIG. 5D.

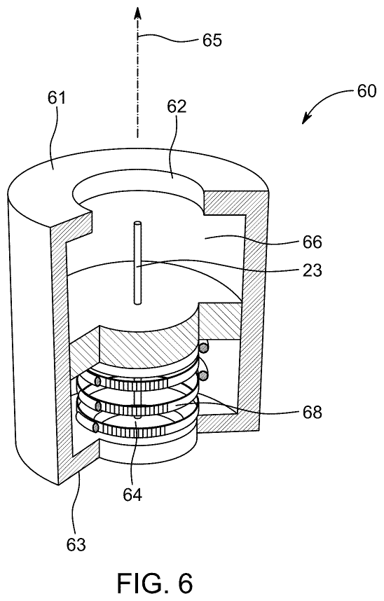

The needle carousel or drum 50 includes a body 51 to be received in a space 111 at the first end 11 of the housing 10. The body 51 is rotatable about an axis 55 substantially parallel to the axis 15 of the housing 10. The body 51 includes multiple needle capsules 60 disposed around the axis 55. FIG. 6 is a cross-sectional view of an example needle capsule 60, according to an embodiment. The needle capsule 60 includes a chamber 66 extending along an axis 65 between a first end 61 and a second end 63 thereof. The chamber 66 has a first opening 62 at the first end 61 and a second opening 64 at the second end 63. A needle 23 is received inside the chamber 66.

The needle 23 extends along the axis 65 and is supported by a needle spring 68 to suspend between the first and second opening 62, 64. The needle spring 68 can be stretched or compressed from its resting position to move the needle 23 along the axis 65 towards the first opening 62 or the second opening 64.

In some cases, the first and second openings 62, 64 of each needle capsule 60 may be hermetically sealed. The needle 23 can be suspended between the hermetically sealed openings 62, 64 to maintain sterility until the point of use. The needle spring 68 can be stretched or compressed from its resting or neutral position to move the needle 23 to pierce the hermetically scaled openings 62, 64 at the point of use (e.g., during priming, or injection).

The needle spring 68 can be stretched or compressed through various mechanism. In an embodiment, a clicker mechanism such as the clicker mechanism 550 of FIGS. 5B-5F may be provided at one end 11 or 13 of the housing 10 and configured to stretch or compress the needle spring 68. When the clicker mechanism is pressed once, the needle spring 68 retracts and the clicker mechanism connects to the needle capsule 60 in a needle carousel. When the clicker mechanism is pressed again, the needle spring 68 is released and the needle 23 is extended.

After injection, the needle 23 can retract back into the needle capsule 60 for easier sharps disposal. The needle carousel 50 can be rotated to allow for the next needle capsule to be utilized. The needle carousel 50 can be replaced with a new one after all the needles being used, which can obviate the need for sharps disposal of spent needles.

The number of needle capsules 60 included by the body 51 of the needle carousel or drum 50 may be two, three, four, five, six, or other suitable numbers. In the embodiment depicted in FIGS. 5A-C, six needle capsules 60 are provided. The second or distal openings 64 of the respective needle capsules 60 are shown in FIG. 5G. The multiple need capsules 60 may be the same or have different sizes. In some cases, the multiple need capsules may receive the needles 23 having substantially the same size or volume. When assembled in the needle carousel or drum 50, each needle capsule 60 may have the axis 65 substantially parallel to the axis 55 of the carousel body 51. In some cases, the axis 55 may be offset from the axis 101 of the housing 10 to allow the axes 65 of each needle capsule 60 being aligned with the axis 101 of the housing 10 by rotating the needle carousel 50 around the axis 55.

The carousel/drum cover 52 includes an inside wall 522 to define a cavity to at least partially receive the needle carousel/drum 50. The carousel/drum cover 52 may have any suitable configurations to mount the needle carousel/drum 50 at the first end 11 of the housing 10 and rotate the needle carousel/drum 50 about the axis 55. For example, in an embodiment, a rotation structure (not shown) may be provided on the inside wall 522 to engage with a rotation structure 54 of the needle carousel/drum 50. The engaged rotation structures 54 may allow multiple stops during the rotation, each of which corresponds to one needle capsule 60 being aligned with the medication cartridge 106 at the first end 11 of the housing 10. As shown in FIG. 5H, the carousel/drum cover 52 further includes an opening 526 on a bottom surface 524 thereof. When the needle carousel/drum 50 rotates with respect to the carousel/drum cover 52, the opening 526 of the carousel/drum cover 52 can be sequentially aligned with the second openings 64 of the respective needle capsules 60.

In some cases, the medication delivery device 500 may be provided with a separate kit that includes two or more needle carousels/drums 50. Each of the needle carousels/drums 50 may include multiple, e.g., six, needle capsules 60. One needle carousel/drum 50 may be replaceably attached to the medication delivery device 500 at a time.

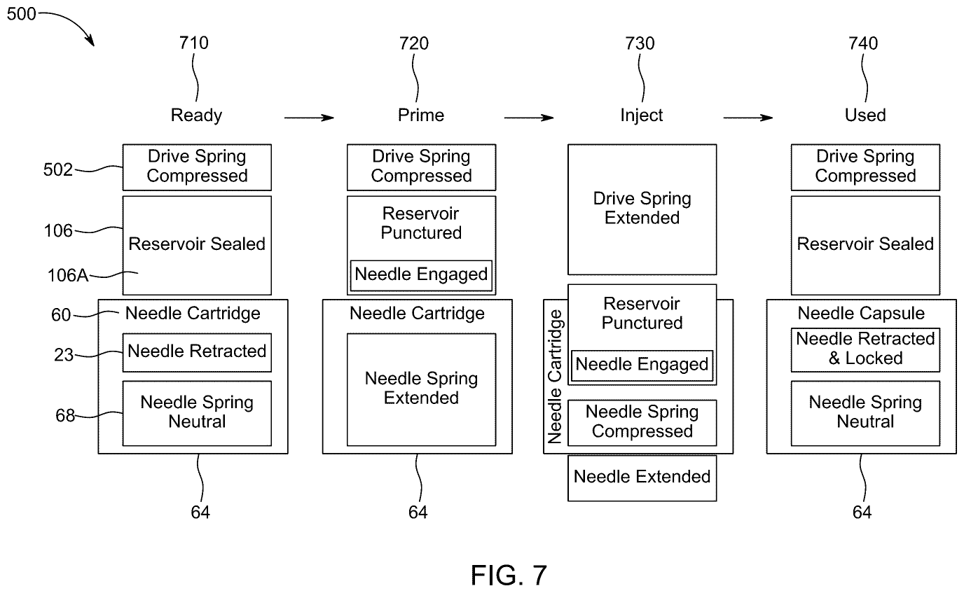

FIG. 7 is a block diagram illustrating the medication delivery device 500 under different stages of operation, according to an embodiment. At stage 710, the medication delivery device 500 operates at a “ready” state. The cartridge spring 502 is at a compressed state to apply a bias force on the medication cartridge 106 toward the first end 11 of the medication delivery device 500. The medication cartridge 106 is in a resting position with its distal end 106a aligned with one of the multiple needle capsules 60 supported by the needle carousel/drum 50. The medication cartridge 106 may have the distal end 106a being hermetically sealed. The spring needle 68 is in a neutral state and the needle 23 is suspended by the spring needle 68 between the first and second openings of the needle capsule 60.

At stage 720, the medication delivery device 500 operates to prime the device. When the medication delivery device 3500 receives, via the user interface or the injection button, a user's instruction, to start an injection, the medication delivery device 500 may automatically prime the device. The needle spring 23 extends from the neutral state toward the medication cartridge 106. The extension of the needle spring 23 can move the needle 23 toward the medication cartridge 106 to engage the distal end 106a of the medication cartridge 106. For example, the needle 23 can penetrate the sealed first opening 62 of the needle capsule 60 and pierce the sealed end 106a of the medication cartridge 106 completely. When the needle 23 is properly engaged with the medication cartridge 106, the medication delivery device 500 can start to prime the device, for example, by operating the drive mechanism to advance the displacement member within the medication cartridge with a pre-determined distance or units of dose.

At stage 730, after the device is primed, the medication delivery device 500 operates to extend the needle 23 to insert and inject medication into a user's skin. When the medication delivery device 500 determines that the priming is completed, the cartridge spring 502 may change from the compressed state to an extended state to move the medication cartridge 106 along with the needle 23 attached thereon toward the second opening 64 of the needle capsule 60. The needle spring 68 is compressed to cause the needle 23 to puncture a hermetic seal at the second opening 64 of the needle capsule 60 and extend through the opening 526 of the carousel/drum cover 52. The needle 23 can be temporarily locked at the extended position with a desired protrusion length by, for example, the locking mechanism 207 of FIG. 2B.

At stage 740, the medication delivery device 500 operates to retract the needle 23 after the user has utilized the needle 23 for injection. The needle spring 60 restores its neutral state and the cartridge spring 502 is compressed to disengage the needle 23 with the medication cartridge 106. The used needle 23 can be locked at the retracted position and be suspended inside the needle capsule 60 for disposal. The user can rotate the needle carousel to have the next needle capsule in the ready state at stage 710. When all the needle capsules have been used, the needle carousel can be replaced with a new one, which can obviate the need for sharps disposal of individual spent needles.

### Aspects:

It is appreciated that any one of aspects can be combined with other aspect(s).
Aspect 1. A medication delivery device comprising:
a housing extending along a housing axis between a distal end and a proximal end thereof; and
a needle cover connected to the distal end of the housing, the needle cover comprising a retractable sheath mechanism axially movable between a first position and a second position,
wherein the retractable sheath mechanism comprises one or more skin sensors disposed on an end surface thereof to detect skin.
   Aspect 2. The medication delivery device of Aspect 1, wherein the needle cover further comprises a locking mechanism to lock the retractable sheath mechanism at the second position.
   Aspect 3. The medication delivery device of Aspect 2, wherein the locking mechanism has a spring-loaded bayonet structure.
   Aspect 4. The medication delivery device of any of Aspects 1-3, wherein the needle cover further comprises an inner collar and an outer collar rotatably connected to the inner collar, the inner collar and the outer collar being axially movable with respect to each other.
   Aspect 5. The medication delivery device of Aspect 4, wherein at least one of the inner collar and the outer collar is connected to the distal end of the housing.
   Aspect 6. The medication delivery device of Aspect 4 or 5, wherein at least one of the inner collar and the outer collar comprises one or more detent features to stop a relative axial movement of the inner collar and the outer collar at a plurality of discrete axial positions representing a plurality of injection depths.
   Aspect 7. The medication delivery device of any of Aspects 1-6, further comprises a needle carousel connected to the distal end of the housing, the needle carousel being rotatable about a carousel axis.
   Aspect 8. The medication delivery device of Aspect 7, wherein the carousel axis is offset from the housing axis.
   Aspect 9. The medication delivery device of Aspect 7 or 8, wherein the needle carousel comprises a plurality of needle capsules disposed around the carousel axis of the needle carousel.
   Aspect 10. The medication delivery device of any of Aspects 7-9, wherein the plurality of needle capsules each comprises a needle and a needle spring to suspend the needle between opposite ends of the respective needle capsules.
   Aspect 11. A method of delivering medication using a medication delivery device, the method comprising:
receiving, via a user interface of the medication delivery device, an instruction to deliver medication into a user's skin;
upon receiving the instruction, measuring, via one or more skin sensors, a contact between a retractable sheath mechanism of the medication delivery device and the user's skin, wherein the retractable sheath mechanism retracts from a first position to a second position to allow a needle of the medication delivery device to insert into the user's skin;
determining, via a controller of the medication delivery device, whether the needle is inserted into the user's skin with a predetermined injection depth based on the measured contact; and
when the needle of the medication delivery device is inserted into the user's skin with the predetermined injection depth, delivering medication into the user's skin, and
when the needle of the medication delivery device is not inserted into the user's skin with the predetermined injection depth, presenting, via the user interface, a feedback to the user to adjust a position of the needle.
   Aspect 12. The method of Aspect 11, wherein the delivering of medication comprises driving a lead screw via an electrical stepper motor.
   Aspect 13. The method of Aspect 11 or 12, further comprising temporarily locking the retractable sheath mechanism at the second position when the needle of the medication delivery device is inserted into the user's skin with the predetermined injection depth.
   Aspect 14. The method of Aspect 13, further comprising releasing the retractable sheath mechanism from the second position to the first position after the delivering of medication into the user's skin.
   Aspect 15. The method of any of Aspects 11-14, further comprising adjusting a protrusion length of the needle with respect to an opening of the retractable sheath mechanism before the needle is inserted into the user's skin, wherein the needle has a distal tip extending away from the opening of the retractable sheath mechanism when the retractable sheath mechanism retracts from the first position to the second position.
   Aspect 16. A method of using a medication delivery device, the method comprising:
receiving, via a user interface of the medication delivery device, an instruction to deliver medication into a user's skin;
upon receiving the instruction, measuring, via one or more skin sensors, a contact between a retractable sheath mechanism of the medication delivery device and the user's skin, wherein the retractable sheath mechanism retracts from a first position to a second position to allow a needle of the medication delivery device to insert into the user's skin;
determining, via a controller of the medication delivery device, whether the needle is inserted into the user's skin with a predetermined injection depth based on the measured contact; and
when the needle of the medication delivery device is not inserted into the user's skin with the predetermined injection depth, presenting, via the user interface, a feedback to the user to adjust a position of the needle.
   Aspect 17. A method of delivering medication using a medication delivery device, the method comprising:
providing a needle carousel to support a plurality of needle capsules disposed around a carousel axis thereof, each needle capsule including a needle spring to suspend a needle between opposite ends of the respective needle capsules;
rotating the needle carousel about the carousel axis to align the needle of one of the needle capsules to an injection end of the medication delivery device;
engaging the needle with the injection end of the medication delivery device;
extending the needle to project from a distal end of the respective needle capsules; and
retracting the needle into the respective needle capsules.
   Aspect 18. A method of using a medication delivery device, the method comprising:
providing a needle carousel to support a plurality of needle capsules disposed around a carousel axis thereof, each needle capsule including a needle spring to suspend a needle between opposite ends of the respective needle capsules;
rotating the needle carousel about the carousel axis to align the needle of one of the needle capsules to an injection end of the medication delivery device;
engaging the needle with the injection end of the medication delivery device;
extending the needle to project from a distal end of the respective needle capsules; and
retracting the needle into the respective needle capsules.
   Aspect 19. The method of Aspect 17 or 18, wherein the engaging of the needle with the injection end further comprises extending the needle spring toward the injection end of the medication delivery device.
   Aspect 20. The method of any of Aspects 17-19, wherein the extending of the needle to project from the distal end further comprises compressing the needle spring away from the injection end of the medication delivery device.
   Aspect 21. The method of any of Aspects 17-20, wherein the retracting of the needle into the respective needle capsules further comprises restoring the needle spring in a neutral position.
   Aspect 22. The method of any of Aspects 17-21, further comprising pressing, via a drive spring, a medication cartridge against the needle carousel that is aligned at the injection end of the medication delivery device.
   Aspect 23. The method of any of Aspects 17-22, further comprising piercing, via the needle, a hermetic sealing at the opposite ends of the respective needle capsules.
   Aspect 24. A medication delivery device comprising:
a user interface for receiving an instruction to deliver medication into a user's skin; and
a needle cover comprising a retractable sheath mechanism axially movable between a first position and a second position configured to allow insertion of a needle into the user's skin, the retractable sheath mechanism comprising one or more skin sensors for detecting the user's skin;
a controller configured to:
   in response to receiving the instruction, measuring, via the one or more skin sensors, a contact between the retractable sheath mechanism and the user's skin;
   determining whether the needle is inserted into the user's skin with a predetermined injection depth based on the measured contact; and
   in response to the needle being inserted into the user's skin with the predetermined injection depth, delivering medication into the user's skin, and
   in response to the needle not being inserted into the user's skin with the predetermined injection depth, presenting, via the user interface, a feedback to the user to adjust a position of the needle.
      Aspect 25. The medication delivery device of Aspect 24, further comprising:
a drive mechanism comprising a lead screw and an electrical stepper motor,
wherein the controller being configured to deliver the medication into the user's skin comprises the controller operating the electrical stepper motor to drive the lead screw.
   Aspect 26. The medication delivery device of Aspect 24 or 25, wherein needle cover includes a locking mechanism for the sheath mechanism, and the controller is configured to, via the locking mechanism, temporarily lock the retractable sheath mechanism at the second position in response to the needle of the medication delivery device being inserted into the user's skin with the predetermined injection depth.
   Aspect 27. The method of Aspect 26, wherein the controller is configured to, via the locking mechanism, release the retractable sheath mechanism from the second position to the first position after the delivering of the medication into the user's skin.
   Aspect 28. The method of any of Aspects 24-27, wherein the controller is configured to:
in response to the needle being inserted into the user's skin with the predetermined injection depth, present, via the user interface, a feedback to the user to adjust a position of the needle.
   Aspect 29. A method of using a medication delivery device, the medication delivery device comprising a needle cover comprising an inner collar, an outer collar moveably connected to the inner collar, and the retractable sheath mechanism moveably connected to the outer collar, the method comprising:
axially moving the retractable sheath mechanism between a first position and a second position to protrude a needle of the medication delivery device from the needle cover, a protrusion length of the needle projecting outside the needle cover in the second position; and
rotating the outer collar relative to the inner collar, wherein the rotating of the outer collar relative to the inner collar adjusts the protrusion length of the needle.
   Aspect 30. The method of Aspect 29, wherein the rotating of the outer collar relative to the inner collar axially adjusts the first position of the retractable sheath mechanism relative to a housing of the medication delivery device.
   Aspect 31. The method of Aspect 29 or 30, wherein the rotating of the outer collar relative to the inner collar axially moves the outer collar and the retractable sheath mechanism relative to a housing of the medication delivery device.
   Aspect 32. The method of any of Aspects 29-31, wherein the needle is mounted on a needle adaptor attached to the inner collar.
   Aspect 33. The method of any of Aspects 30-32, wherein threads rotatably connect the outer collar and the inner collar.
   Aspect 34. The method of any of Aspects 29-33, wherein the rotating the outer collar relative to the inner collar includes axially moving the outer collar between a plurality of discreate axial positions representing a plurality of injection depths, at least one of the inner collar and the outer collar comprises one or more detent features to stop the axial movement of the outer collar at the plurality of discreate axial positions.
   Aspect 35. A medication delivery device comprising:
a housing extending along a housing axis between a distal end and a proximal end thereof;
a needle cover connected to the distal end of the housing, the needle cover comprising:
   a retractable sheath mechanism axially movable between a first position and a second position, the retractable sheath mechanism in the second position configured to project a protrusion length of a needle outside of the needle cover,
   an inner collar; and
   an outer collar rotatably connected to the inner collar to axially move the outer collar relative to the inner collar, the axial movement of the outer collar relative to the inner collar configured to adjust the protrusion length of the needle.
      Aspect 36. The medication delivery device of Aspect 35, wherein at least one of the inner collar and the outer collar is connected to the distal end of the housing.
      Aspect 37. The medication delivery device of Aspect 35 or 36, wherein at least one of the inner collar and the outer collar comprises one or more detent features to stop a relative axial movement of the inner collar and the outer collar at a plurality of discrete axial positions representing a plurality of injection depths.
      Aspect 38. The medication delivery device of any of Aspects 35-37, further comprising a needle carousel connected to the distal end of the housing, the needle carousel being rotatable about a carousel axis, the needle carousel configured to provide the needle.
      Aspect 38. The medication delivery device of Aspect 38, wherein the needle carousel comprises a plurality of needle capsules disposed around the carousel axis of the needle carousel, and the plurality of needle capsules each comprises a needle and a needle spring to suspend the needle between opposite ends of the respective needle capsules.

The terminology used in this specification is intended to describe particular embodiments and is not intended to be limiting. The terms “a,” “an,” and “the” include the plural forms as well, unless clearly indicated otherwise. The terms “comprises” and/or “comprising,” when used in this specification, specify the presence of the stated features, integers, steps, operations, elements, and/or components, but do not preclude the presence or addition of one or more other features, integers, steps, operations, elements, and/or components.

With regard to the preceding description, it is to be understood that changes may be made in detail, especially in matters of the construction materials employed and the shape, size, and arrangement of parts without departing from the scope of the present disclosure. This specification and the embodiments described are exemplary only, with the true scope and spirit of the disclosure being indicated by the claims that follow.

1. A medication delivery device comprising:
a housing extending along a housing axis between a distal end and a proximal end thereof; and
a needle cover connected to the distal end of the housing, the needle cover comprising a retractable sheath mechanism axially movable between a first position and a second position,
wherein the retractable sheath mechanism comprises one or more skin sensors disposed on an end surface thereof to detect skin.

2. The medication delivery device of claim 1, wherein the needle cover further comprises a locking mechanism to lock the retractable sheath mechanism at the second position.

3. The medication delivery device of claim 2, wherein the locking mechanism has a spring-loaded bayonet structure.

4. The medication delivery device of claim 1, wherein the needle cover further comprises an inner collar and an outer collar rotatably connected to the inner collar, the inner collar and the outer collar being axially movable with respect to each other.

5. The medication delivery device of claim 4, wherein at least one of the inner collar and the outer collar is connected to the distal end of the housing.

6. The medication delivery device of claim 4, wherein at least one of the inner collar and the outer collar comprises one or more detent features to stop a relative axial movement of the inner collar and the outer collar at a plurality of discrete axial positions representing a plurality of injection depths.

7. The medication delivery device of claim 1, further comprising a needle carousel connected to the distal end of the housing, the needle carousel being rotatable about a carousel axis.

8. The medication delivery device of claim 7, wherein the carousel axis is offset from the housing axis.

9. The medication delivery device of claim 7, wherein the needle carousel comprises a plurality of needle capsules disposed around the carousel axis of the needle carousel.

10. The medication delivery device of claim 7, wherein the plurality of needle capsules each comprises a needle and a needle spring to suspend the needle between opposite ends of the respective needle capsules.

11. A medication delivery device comprising:
a user interface for receiving an instruction to deliver medication into a user's skin;
a needle cover comprising a retractable sheath mechanism axially movable between a first position and a second position configured to allow insertion of a needle into the user's skin, the retractable sheath mechanism comprising one or more skin sensors for detecting the user's skin;
a controller configured to:
in response to receiving the instruction, measuring, via the one or more skin sensors, a contact between the retractable sheath mechanism and the user's skin;
determining whether the needle is inserted into the user's skin with a predetermined injection depth based on the measured contact; and
in response to the needle being inserted into the user's skin with the predetermined injection depth, delivering medication into the user's skin, and
in response to the needle not being inserted into the user's skin with the predetermined injection depth, presenting, via the user interface, a feedback to the user to adjust a position of the needle.

12. The medication delivery device of claim 11, further comprising:
a drive mechanism comprising a lead screw and an electrical stepper motor,
wherein the controller is configured to deliver the medication into the user's skin comprises the controller operating the electrical stepper motor to drive the lead screw.

13. The medication delivery device of claim 11, wherein needle cover includes a locking mechanism for the sheath mechanism, and the controller is configured to, via the locking mechanism, temporarily lock the retractable sheath mechanism at the second position in response to the needle of the medication delivery device being inserted into the user's skin with the predetermined injection depth.

14. The medication delivery device of claim 13, wherein the controller is configured to, via the locking mechanism, release the retractable sheath mechanism from the second position to the first position after the delivering of the medication into the user's skin.

15. The medication delivery device of claim 11, wherein the controller is configured to:
in response to the needle being inserted into the user's skin with the predetermined injection depth, present, via the user interface, a feedback to the user to adjust a position of the needle.

16. A method of using a medication delivery device, the method comprising:
providing a needle carousel to support a plurality of needle capsules disposed around a carousel axis thereof, each needle capsule including a needle spring to suspend a needle between opposite ends of the respective needle capsules;
rotating the needle carousel about the carousel axis to align the needle of one of the needle capsules to an injection end of the medication delivery device;
engaging the needle with the injection end of the medication delivery device;
extending the needle to project from a distal end of the respective needle capsules; and
retracting the needle into the respective needle capsules.

17. The method of claim 16, wherein the engaging of the needle with the injection end further comprises extending the needle spring toward the injection end of the medication delivery device.

18. The method of claim 16, wherein the extending of the needle to project from the distal end further comprises compressing the needle spring away from the injection end of the medication delivery device.

19. The method of claim 16, wherein the retracting of the needle into the respective needle capsules further comprises restoring the needle spring in a neutral position.

20. The method of claim 16, further comprising:
pressing, via a drive spring, a medication cartridge against the needle carousel that is aligned at the injection end of the medication delivery device; and
piercing, via the needle, a hermetic sealing at the opposite ends of the respective needle capsules.
